(19) United States
(12) Patent Application Publication
(10) Pub. No.: US 2025/0303419 A1
(43) Pub. Date: Oct. 2, 2025

(54) REACTANT ARRAY DEVICES, SYSTEMS, AND METHODS

(71) Applicant: SENSILL, INC., Concord, CA (US)

(72) Inventor: William Shea, Martinez, CA (US)

(73) Assignee: SENSILL, INC., Concord, CA (US)

(21) Appl. No.: 19/229,780

(22) Filed: Jun. 5, 2025

Related U.S. Application Data
(63) Continuation of application No. PCT/US2023/083104, filed on Dec. 8, 2023.

(51) Int. Cl. B01L 3/00; G01N 33/52

(52) U.S. Cl. CPC B01L 3/502761; G01N 33/528; B01L 2200/0647; B01L 2200/12; B01L 2300/0663

(57) ABSTRACT

Devices, systems, and methods include a reactant array. The reactant array may include a sintered material and a reactant material applied to the sintered material. The reactant material may be configured to change one or more properties in response to exposure to one or more analytes. The reactant array may be used to identify an analyte of a fluid to which the reactant material is exposed based on a change in one or more properties of the reactant material in response to exposure to the fluid. The reactant array may be applied to a multi-dimensional object and passively and/or actively exposed to the fluid.

### CROSS-REFERENCE TO RELATED APPLICATIONS

This application is a continuation of International Application No. PCT/US2023/083104, filed Dec. 8, 2023, which claims priority to: U.S. Provisional Patent Application Ser. No. 63/431,507, filed Dec. 9, 2022, the entirety of which is incorporated herein by reference; U.S. Provisional Patent Application Ser. No. 63/431,510, filed Dec. 9, 2022, the entirety of which is incorporated herein by reference; U.S. Provisional Patent Application Ser. No. 63/431,519, filed Dec. 9, 2022, the entirety of which is incorporated herein by reference; U.S. Provisional Patent Application Ser. No. 63/431,525, filed Dec. 9, 2022, the entirety of which are incorporated herein by reference; U.S. Provisional Patent Application Ser. No. 63/431,528, filed Dec. 9, 2022, the entirety of which are incorporated herein by reference; U.S. Provisional Patent Application Ser. No. 63/431,533, filed Dec. 9, 2022, the entirety of which are incorporated herein by reference.

### TECHNICAL FIELD

The present disclosure pertains to sensing and analysis tools, and the like. More particularly, the present disclosure pertains to devices and systems for sensing and analyzing chemical substances, and methods for manufacturing and using such devices.

### BACKGROUND

A wide variety of devices have been developed for collection, storing, sensing, and analysis of samples. These devices are manufactured by any one of a variety of different manufacturing methods and may be used according to any one of a variety of methods. Of the known medical devices and methods, each has certain advantages and disadvantages.

### BRIEF SUMMARY

This disclosure provides design, material, manufacturing method, and use alternatives for sensing and analysis devices. Although it is noted that collection, storing, sensing, and analysis approaches and systems are known, there exists a need for improvement on those approaches and systems.

An example colorimetric sensor array including a substrate, a sintered material, and a reactant material applied to the sintered material, and wherein the reactant material is configured to change one or more properties in response to exposure to one or more volatile compounds or gases.

Alternatively or additionally to any of the embodiments in this section, the reactant material may be applied to an entirety of a surface area of the sintered material.

Alternatively or additionally to any of the embodiments in this section, the reactant material may be applied to a desired portion of a surface area of the sintered material.

Alternatively or additionally to any of the embodiments in this section, the sintered material and the reactant material applied to the sintered material may form a coated carrier and the coated carrier may be secured to the substrate.

In another example, a method of forming a colorimetric sensor array may include applying a reactant material to a sintered material, allowing the reactant material to dry on the sintered material to form a coated carrier, and secure the coated carrier to a substrate.

Alternatively or additionally to any of the embodiments in this section, the applying reactant material to the sintered material may include applying the reactant material to the sintered material using an electrostatic deposition technique.

Alternatively or additionally to any of the embodiments in this section, the applying reactant material to the sintered material may include impregnating the sintered material with an aqueous chemical solution.

In another example, a colorimetric sensor array may include a three-dimensional object, and a reactant material applied to the three-dimensional object, and wherein the reactant material is configured to change one or more properties in response to exposure to one or more volatile compounds or gases.

Alternatively or additionally to any of the embodiments in this section, the three-dimensional object may be a bandage.

Alternatively or additionally to any of the embodiments in this section, the three-dimensional object may be a living plant.

Alternatively or additionally to any of the embodiments in this section, the three-dimensional object may be a sample testing cup.

In another example, a method of forming a colorimetric sensor array may include applying a reactant material to a multi-dimensional object, and wherein the reactant material is configured to change one or more properties in response to exposure to one or more volatile compounds or gases.

Alternatively or additionally to any of the embodiments in this section, the multi-dimensional object may be skin on a subject.

Alternatively or additionally to any of the embodiments in this section, the multi-dimensional object may be a bandage configured to be applied to a wound of a subject.

Alternatively or additionally to any of the embodiments in this section, the multi-dimensional object may be a flower.

Alternatively or additionally to any of the embodiments in this section, the multi-dimensional object may be a sample testing cup.

Alternatively or additionally to any of the embodiments in this section, the multi-dimensional object may be a liquid.

Alternatively or additionally to any of the embodiments in this section, the applying the reactant to the multi-dimensional object may include sprinkling the reactant material on the multi-dimensional object.

In another example, a reactant array may include a sintered material and a reactant material applied to the sintered material, wherein the reactant material may be configured to change one or more properties in response to exposure to one or more analytes.

Alternatively or additionally to any of the embodiments in this section, the reactant material may be applied to an entirety of a surface area of the sintered material.

Alternatively or additionally to any of the embodiments in this section, the reactant material may be applied to a desired portion of a surface area of the sintered material.

Alternatively or additionally to any of the embodiments in this section, the reactant array may further include a substrate, wherein the sintered material and the reactant material may be applied to the sintered material form a coated carrier and the coated carrier is secured to the substrate.

Alternatively or additionally to any of the embodiments in this section, the substrate may include a filter.

Alternatively or additionally to any of the embodiments in this section, the reactant array may further include a three-dimensional object, wherein the sintered material and the reactant material may be applied to the sintered material form a coated carrier and the coated carrier is applied to the three-dimensional object.

Alternatively or additionally to any of the embodiments in this section, the three-dimensional object may include a bandage.

Alternatively or additionally to any of the embodiments in this section, the three-dimensional object may include a living plant.

Alternatively or additionally to any of the embodiments in this section, the three-dimensional object may include a sample testing cup.

Alternatively or additionally to any of the embodiments in this section, the three-dimensional object may include a filter.

In another example, a method of forming a reactant array including applying a reactant material to a sintered material, drying the reactant material on the sintered material to form a coated carrier, and applying the coated carrier to a multi-dimensional object.

Alternatively or additionally to any of the embodiments in this section, the applying the reactant material to the sintered material may include applying the reactant material to the sintered material using an electrostatic deposition technique.

Alternatively or additionally to any of the embodiments in this section, the applying the reactant material to the sintered material may include impregnating the sintered material with an aqueous chemical solution.

Alternatively or additionally to any of the embodiments in this section, the multi-dimensional object may include a bandage.

Alternatively or additionally to any of the embodiments in this section, the multi-dimensional object may include a living plant.

Alternatively or additionally to any of the embodiments in this section, the multi-dimensional object may include a sample testing cup.

Alternatively or additionally to any of the embodiments in this section, the multi-dimensional object may include a sample testing cup.

In another example, a method of testing fluid may include applying a coated carrier to a multi-dimensional object, exposing the coated carrier to a fluid, observing the coated carrier over time after initial exposure of the coated carrier to the fluid, and identifying an analyte of the fluid based on a change in a property of one or more properties of the coated carrier in response to exposure to the fluid.

Alternatively or additionally to any of the embodiments in this section, the coated carrier may comprise a sintered material to which a reactant material is applied.

Alternatively or additionally to any of the embodiments in this section, the multi-dimensional object may include skin on a subject.

Alternatively or additionally to any of the embodiments in this section, the multi-dimensional object may include a bandage configured to be applied to a wound of a subject.

Alternatively or additionally to any of the embodiments in this section, the multi-dimensional object may include a sample testing cup.

Alternatively or additionally to any of the embodiments in this section, applying the coated carrier to the multi-dimensional object may include sprinkling the coated carrier on the multi-dimensional object.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative sensing system;

FIG. 2 is an electron micrograph image of a porous sintered polymer material;

FIGS. 3A-3E schematically depict illustrative three-dimensional shapes of coated carriers;

FIG. 4 is a schematic diagram of an illustrative reactant array view of an illustrative sensing system;

FIGS. 5A and 5B schematically depict a perspective view and an end view of an illustrative reactant array;

FIGS. 6A-6E schematically depict illustrative reactant arrays;

FIG. 7 schematically depicts an illustrative technique for forming a reactant array; and

FIG. 8 schematically depicts an illustrative technique for analyzing fluid.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

The term “fluid” is inclusive of both liquids and gases.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Fluids with concentrations of analytes such as volatile compounds (e.g., volatile organic compounds (VOCs)) and/or gases, which may or may not be hazardous, may be sensed, analyzed, and/or monitored. Sensing, analyzing, and/or monitoring of fluids with analytes (e.g., non-volatile and/or volatile compounds, gases, and/or other fluids) may utilize absorption measurements of reactants exposed to such fluids for any purpose including, but not limited to, diagnostic hazard warning, manufacturing processes or quality control, record keeping, archival purposes, product development, product-consumer matching, etc.

In some cases, VOCs and/or gases may be present in ambient fluid (e.g., ambient air, etc.) and sensed, analyzed, and/or monitored using reactants for real-time alarms, to treat subjects, or to collect and/or archive data for health records, regulatory compliance records, etc. Further, VOCs and/or gases exhaled or emitted, excreted, emanated, released, and/or secreted from a subject (e.g., humans, mammals, animals other than humans or mammals, food, produce, meat, pathogens, bacteria (e.g., good and/or bad bacteria), plants, wounds, ulcers, surgical sites, skin of a subject, mouth of a subject, nasal passages of a subject, sinuses of a subject, rectum area of a subject, vaginal area of a subject, genitals area of a subject, ear canals of a subject, pores of a subject, etc.) may be sensed, analyzed, and/or monitored to assess hazardous, dangerous, or illegal substances in or at the subject or target site, a lung condition of lungs of a subject, a condition of a blood disease, a condition of infections, conditions related to diseases or biological conditions, conditions related to general health, conditions related to food flavors, conditions related to perfumes or smells, and/or other suitable conditions.

The systems discussed herein for sensing, analyzing, and/or monitoring fluids (e.g., for analytes of interest) may be configured to accurately detect and record a reactant array response (e.g., a colorimetric sensor array (CSA) spectral response) to exposure to the fluids. The systems may utilize techniques for non-invasively detecting one or more analytes of interest (e.g., one or more pathogens responsible for specific human skin infections including, but not limited to, skin infections, urinary tract infections (UTIs), vaginitis, wound infections, ulcers, etc., and/or other suitable analytes) from a fluid using a reactant array to allow for early detection of and early implementation of protocols to address one or more conditions associated with any sensed analytes of interest. In one example, enhanced classification of one or more analytes using the systems described herein may enable detection and identification of responsible pathogens at the very beginning stages of a dangerous skin infection, which may result in a high level of protection and probability of a favorable outcome for subjects.

An analysis system (e.g., a reactant array analysis system) may include an enclosure or cartridge containing a reactant array (e.g., a CSA having a reactant array) and a device for reading or otherwise analyzing the reactant array (e.g., a reader device) in the enclosure or cartridge before, during, and/or after the reactant array is exposed to fluids from a target area or sample of a target area. When included, the enclosure or cartridge may facilitate exposing the reactant array to fluid and/or positioning the reactant array relative to the device for reading and/or analyzing the reactant array. The enclosure or cartridge, however, may be omitted and the reactant array may be exposed to a fluid and analyzed with the device without a need for the cartridge. Alternatively or additionally, the device may be omitted and changes in the reactant array may be observed by a user (e.g., with the naked eye and/or via the naked eye with one or more lenses)

The device for analyzing the reactant array may be or may include a spectrometer, an image sensor, and/or other suitable image or light collectors/sensors (e.g., configured to facilitate computer vision). In some instances, the device may be configured to be a handheld device or a benchtop device. Further, the device for analyzing the reactant array may be configured to be simple to use, minimize human error, reduce cross contamination of samples, and reduce human risk of exposure to analytes received at the device.

Analysis of the reactant array, which may or may not be located in an enclosure or cartridge, may include analyzing the reactant array prior to exposure to the fluid, during exposure to the fluid, and/or after exposure to the fluid is complete. In some examples, the enclosure or cartridge, when included, may contain a reactant array and may be placed into the device for analyzing the reactant array (e.g., via an opening in the device) during and/or through pre and post exposure to fluid for optimal reactant array image/reflectometer analysis. Once the analysis of the reactant array is complete, the reactant array may be removed from the device for analyzing the reactant array and discarded. Having the reactant array be disposable may prevent cross-contaminations of the reactant array and erroneous analyses (e.g., due to the previous fluids persisting in the tubing and reflowing over and/or through the reactant array, undesirably flowing onto a human, etc.).

The reactant array may be formed in any suitable manners. In some examples, the reactant array may be formed by applying (e.g., via printing, spraying, and/or applying with other suitable techniques) chemical formulations (e.g., chemical formulations may be or include chemically impregnated items (CII) in an aqueous or powder state) onto a substrate or membrane. After applying the chemical formulation on the substrate or membrane, the substrates or membranes may be dried, which may drive off a solvent and leave a chemical precipitate on the substrate or membrane that is a desired chemical formulation configured to interact with analytes of interest (e.g., a chemical formulation configured to interact/react with analytes of interest on a molecule-by-molecule basis). Further, the chemical formulations applied to the substrate or membrane and the chemical precipitate that remains after the substrate or membrane dries may be configured to detect analytes that pass over and/or through the chemical formulation on the substrate or membrane.

The substrate or membrane to which the chemical formulations may be applied may be any suitable substrate having one or more surfaces that may receive the chemical formulations. For example, the substrate or membrane may be any multi-dimensional (e.g., two-dimensional (2D), three-dimensional (3D), etc.) object having a surface configured to receive the chemical formations. Example multi-dimensional objects that may have a surface suitable for receiving the chemical formulations may be or may include, but are not limited to, sample cups (e.g., a compartment in a urinary tract infection test cup, etc.), human skin, bandages configured to be applied to a wound or biological material, sintered materials, inert materials (e.g., polyethylene, polypropylene, etc.), polymer materials, metal materials, solids, liquids and/or other suitable substrates or membranes. In some examples, the substrate or membrane to which the chemical formulations configured to detect analytes are applied may be a surface to be analyzed. In one example, the chemical formulations may be applied (e.g., sprinkled, sprayed, etc.) to a substrate that is a surface of a target area (e.g., a wound, skin, a flower (e.g., a living flower or a dead flower), a liquid, a solid, etc.) to determine if an analyte of interest (e.g., a bacteria, a prohibited substance, smell inducing spore, etc.) is present at the target area.

The chemical formulations may be configured in any suitable manner on the substrate or membrane and form reactants configured to interact with (e.g., change one or more properties in response to exposure to) a predetermined analyte. In some cases, the chemical formulations on the substrate or membrane may form reactants that take a form of color bars, color dots, predetermined patterns, and/or other suitable reactant forms. In one example, the chemical formulations may be applied on a flat or substantially flat substrate or membrane configured to be used in a cartridge, where the chemical formulations may be color bars having a rectangular shape and dimensions of approximately 0.5 millimeters (mm) by 3.0 mm for an area of 1.5 mm². Other suitable dimensions are contemplated.

In some cases, it may be desirable to have more surface area than less surface area of the reactant on a substrate or membrane. That is, because primary interactions between an applied reactant and analytes in a fluid occur at a surface of the applied reactant, an increase in a surface area of the applied reactant may result in an increased sensitivity, an increased detection rate, and an increased effectiveness of the reactant on the substrate or membrane.

To facilitate increasing a surface area of a reactant relative to reactants on a flat or substantially flat substrate or membrane, chemical substances may be applied onto a sintered material to form a coated carrier and then the coated carrier may be applied to a substrate or membrane to form the reactant array for use in detection of an analyte. Alternatively or additionally, the sintered material may be the substrate or membrane to which the chemical substances are applied such that the coated carrier may be the reactant array.

Due to an overall increased porosity of sintered material relative to a flat or substantially flat substrate or membrane, the sintered material may provide more surface area per given volume of an applied chemical formulation (e.g., reactant or reactant material) than flat or substantially flat substrates or membranes at which the chemical formulation is applied thereto to interact or react with analytes. Further, a pore size or porosity of a sintered material may be adjusted, as desired, for optimizing an overall reactant surface area, reactant surface area per given volume of reactant, and/or sensitivity of the reactant array.

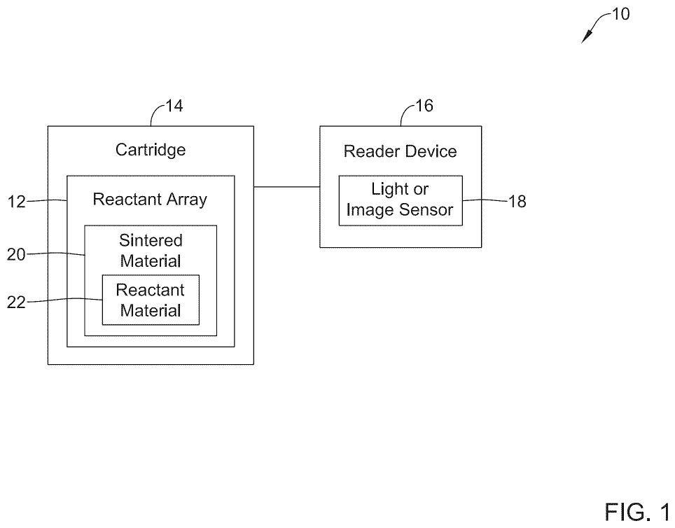

Turning to the Figures, FIG. 1 schematically depicts an illustrative analysis system 10 for analyzing a response of a reactant array 12 to analyte (e.g., non-volatile and/or volatile compounds, gases, liquids, and/or other fluids) in fluids. In some configurations the reactant array 12 may be positioned in, on, or at a cartridge 14 configured to facilitate analysis of the reactant array 12 by a reader device 16, where the reader device 16 may be configured to collect and/or analyze light and/or images from the reactant array 12. In some configurations of the system 10, the cartridge 14 may be omitted and the reader device 16 may collect light from the reactant array 12 without use of the cartridge 14.

The cartridge 14, when included, may have any suitable configuration and may include any suitable components configured to facilitate passing fluid from a target area or a sample from the target area to the reactant array 12 and that may facilitate the reader device 16 collecting light from the reactant array 12. In some configurations, the cartridge 14 may include one or more reactant arrays 12, one or more openings or transparent portions through which the reader device 16 may receive light from the reactant array 12, one or more compartments for housing the reactant array 12, one or more compartments for receiving a sample from a target area, one or more tubes for carrying fluid from the target area or from a sample of a target area to the reactant array, one or more pumps or portions of pumps for passing fluid from the target area or a sample thereof over and/or through the reactant array, and/or other suitable components. Although other suitable configurations of the cartridge 14 are contemplated, illustrative configurations of the cartridges are disclosed in PCT Application No. PCT/US2023/083076 (Attorney docket no. 1519.1009111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes, and PCT Application No. PCT/US2023/083073 (Attorney docket no. 1519.1007111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes.

The reader device 16 may include one more suitable components for reading and/or analyzing the reactant array 12, which may be in or separate from the cartridge 14. Example suitable components of the reader device 16 include, but are not limited to, illumination components, light collection components, one or more light or image sensors 18, one or more cartridge detectors, one or more controllers, one or more motors, one or more light sources, one or more sets of lenses, one or more pumps, one or more buttons, one or more user interfaces, one or more displays, and/or other suitable components. In some examples, the reader device 16 may be a bench top device or a handheld device, each configured to be used with or without the cartridges 14 to analyze the reactant arrays 12. Although other suitable configurations of the reader device 16 are contemplated, illustrative configurations of devices or systems for collecting light from the reactant array 12 and analyzing the light collected are disclosed in PCT Application No. PCT/US2023/083024 (Attorney docket no. 1519.1004111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes, PCT Application No. PCT/US2023/083063 (Attorney docket no. 1519.1005111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes, and PCT Application No. PCT/US2023/083068 (Attorney docket no. 1519.1006111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes.

The light or image sensor 18 may be and/or may include one or more light collectors of any suitable type. Example suitable types of light collectors may include, but are not limited to, a light sensor, an image sensor, an n-dimensional sensor array (e.g., where “n” equals 1, 2, etc.), a linear 2D light detector array image sensor, light detector array image sensor, a spectrometer, a charge-coupled device (CCD) image sensor, complementary metal-oxide semiconductor (CMOS) image sensor, contact image sensor (CIS), color contact image sensor (CCIS), a camera, other suitable light collectors, and/or combinations of light collectors. In one example, the light collector may include or may be a spectrometer configured to measure photons collected from (e.g., reflected, transmitted, and/or otherwise received from) the reactant array. Utilizing a spectrometer may facilitate sensing wavelengths of light with high resolution in the nanometer range and may provide a continuous set of data over the wavelength range, which allows for a sensitive analysis of the data to identify components of a fluid to which the reactant array 12 was exposed relative to when other light collectors are used. In another example, the light collector may include a 2D pixel array image sensor configured to record multiple spatial interferograms in a pixel array direction of an interferogram representing a Fourier transform of the reactant array 12, which may provide sufficient sensitivity, while being compact and cost-effective.

The reactant array 12 may have any suitable configuration. In some configurations, the reactant array 12 may include sintered material 20 and reactant material 22, where the reactant material 22 may be applied to sintered material 20 (e.g., to at least part of the surface of the sintered material). The reactant material 22 may be any suitable material configured to sense one or more analytes (e.g., the reactant material 22 may be an analyte sensitive material) and change one or more properties in response to exposure to the one or more analytes the reactant material 22 is configured to sense. In some examples, the one or more properties of the reactant material 22 that may change in response to detecting the one or more analytes may include a color of the reactant material 22, a transparency of the reactant material 22, a density of the reactant material 22, and/or other suitable properties of the reactant material 22.

The reactant material 22 may be reversible (e.g., reusable), semi-reversible, or non-reversible (e.g., single use). In some examples, the reactant material 22 may be an optically responsive chemical material (e.g., a chemoresponsive material) that changes color in response to detecting one or more analytes in a fluid to which the reactant material 22 is exposed, but other suitable material is contemplated. Example suitable reactant materials 22 include dyes from, but not limited to, the following classes: Lewis acid/base dyes (e.g., metal containing dyes), Brensted acidic or basic dyes (e.g., pH indicators), dyes with large permanent dipoles (e.g., solvatochromic dyes), redox responsive dyes (e.g., metal nanoparticle precursors), and/or other suitable classes of dyes. One example reactant material 22 may be a silver nanoparticle material. Other suitable reactant materials 22 are contemplated, including reactant material 22 other than a printed dye or an optically responsive chemical material.

The reactant array 12 may include one or more reactant materials 22 applied to any desired portion of the surface area of one or more structures of the sintered material 20. In some examples, a plurality of different reactant materials 22 that may or may not react differently to different analytes in fluid to which the reactant materials 22 are exposed may be applied to an entirety of or at least part of a surface area of a single structure of sintered material 20. In some examples, a single reactant material 22 may be applied to an entirety of or at least part of the surface area of a single structure of sintered material 20. In some examples, one or more reactant materials 22 that may or may not react differently to different analytes in fluid to which the reactant materials 22 are exposed may be applied to an entirety of or at least part of the surface areas of a plurality of structures of sintered material 20.

The reactant materials 22 may be applied to the sintered material 20 randomly and/or to form one or more designs or patterns at any desired portion(s) of the sintered material 20. Example configurations of the reactant materials applied to the sintered material 20 include, but are not limited to, bars, circles, lines, crosses, wording, lettering, numbers, punctuation, pictures, artistic designs, diagonals, grid patterns of rows and columns, concentric rings, color matching of a color of reactant material 22 with a color of the sintered material 20 prior to interactions of the reactant material 22 with analyte, patterns that result in identifiable shapes when the reactant material reacts to a particular analyte, other suitable configurations, and/or combinations thereof. In one example, two reactant materials 22 may be applied to one or more structures of the sintered material 20 in alternating fashion along a width of the sintered material 20, but other suitable configurations are contemplated.

The sintered material 20 may be formed from any suitable material configured to facilitate the reactant material 22 adhering thereto and/or reacting to analytes in a fluid to which the reactant material 22 is exposed. Example suitable types of sintered material 20 may include, but are not limited to, polymer materials, metallic materials, ceramic materials, inert materials, thermoplastic materials, polyethylene, polypropylene, polytetrafluoroethylene (PTFE), polyvinylidene Flouride (PVDF), and/or other suitable materials. In some examples, the sintered material may be or may include a sintered polyethylene material, but other suitable materials are contemplated.

The sintered material 20 may be formed from one or more powered materials heated below its melting point to form a coherent mass, without fully melting the powdered materials. A pore size or porosity and/or surface texture of the sintered material 20 may be adjusted during the manufacturing process, as desired, to optimize an overall reactant surface area, reactant surface area per given volume of reactant, and/or sensitivity of the reactant array 12. In some examples, the sintered material 20 of a first material type may have a porosity and/or surface texture that is greater than a porosity of a non-sintered material of the first material type, which may provide a greater surface area per given volume of the reactant material(s) 22 applied to the sintered material 20 relative to when the given volume of reactant material(s) is applied to the non-sintered material.

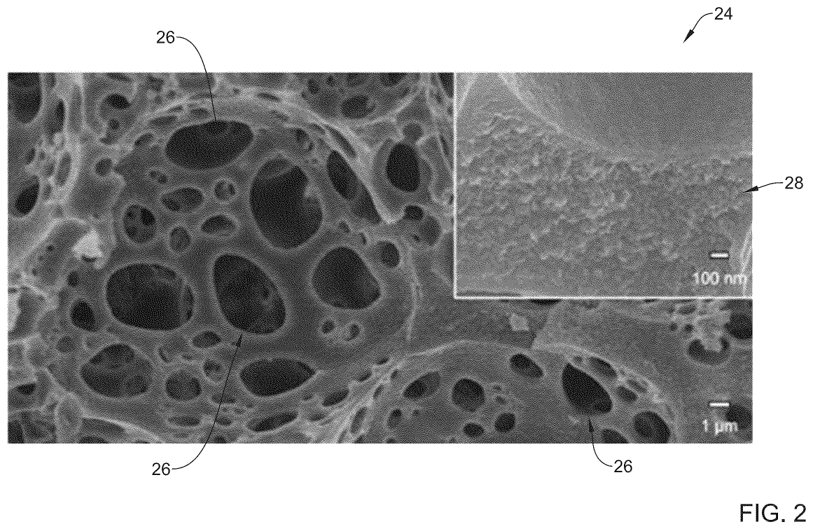

FIG. 2 depicts an electron micrograph image 24 of a porous sintered polymer material that demonstrates illustrative pores in or of and/or surface texture of the sintered material 20 that form surfaces to which the reactant material 22 may be applied so as to increase a surface area per volume of reactant material 22 relative to when the same volume of reactant material 22 is applied to non-sintered material. As depicted in FIG. 2, for example, sintered material 20 formed from polymer material may have pores 26 sized on the micron scale and surface texture 28 on the nanometer scale. Pores 26 having other suitable sizes (e.g., diameters, widths, etc.) and surface textures 28 on other suitable scales are contemplated and may be selected based on properties of the type of material forming the sintered material 20, properties of the reactant material(s) 20, properties of the fluid to be analyzed, and/or based on one or more other suitable factors.

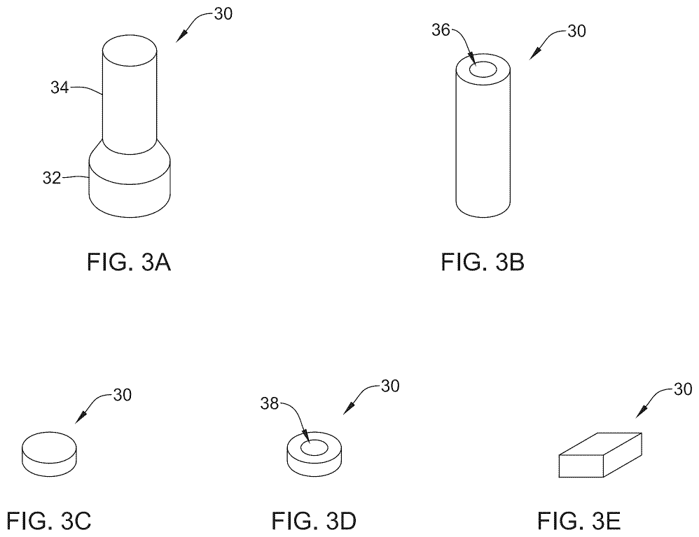

Structures of the sintered material 20 may take on any suitable multi-dimensional shape. For example, suitable structures of the sintered material 20 may include, but are not limited to, sheet structures, powder particles, granular material, beads, elongated structures, ball-shaped structures, pyramid structures, circular structures, annular structures, disc structures, filter structures, and/or other suitable structures. Although other shapes and configurations are contemplated, FIGS. 3A-3E depict illustrative three-dimensional (3D) structures 30 of sintered material 20, which may include the structure 30 having a wide base 32 with an elongated top 34 extending from the wide base 32 (FIG. 3A), the structure 30 having a tube configuration with a lumen 36 extending at least partially therethrough (FIG. 3B), the structure 30 having a disc-shape (FIG. 3C), the structure 30 having an annular or ring shape with an opening 38 in the middle (FIG. 3D), and the structure 30 having a 3D rectangular shape (FIG. 3E).

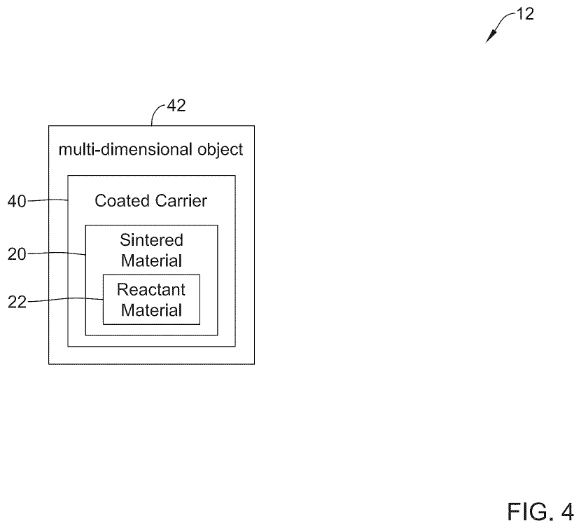

FIG. 4 schematically depicts an illustrative diagram of the reactant array 12 (e.g., a colorimetric sensor (CSA), etc.). The reactant array 12 may include, among other components, one or more coated carriers 40 and one or more 3D objects 42 (e.g., a substrate) to which the coated carrier 40 may be applied and/or supported. Alternatively or additionally, the reactant array 22 may include the one or more coated carriers 40 and the 3D object 42 may be omitted. When the 3D object 42 is included, the coated carrier(s) 40 may be secured to the 3D object and/or supported by the 3D object in an unsecured manner.

The coated carrier 40 may have any suitable configuration and may include the sintered material 20 and the reactant material 22. Although other configurations are contemplated, the coated carrier 40 may take on the form of the structure 30 of the sintered material 20. In some examples, the sintered material 20 may be considered a substrate for the reactant material 22. The reactant material 22 of the coated carrier 40 may entirely or at least partially cover the sintered material 20.

The 3D object 42 to which the coated carrier 40 is optionally applied may be any suitable multi-dimensional object or structure that facilitates exposing the coated carrier 40 to fluids for analysis. For example, the 3D object 42 may take on any suitable shape and/or configuration including, but not limited to, a tube, a membrane, a diaphragm, an elongated support, a cup, a lid of or for a cup, test strip, a bandage, band (e.g., a headband, sweat band, etc.) skin of a subject (e.g., a human, mammal, and/or other animals), a wound of a subject, a living plant, a dead plant, a flower, soil, polymer material, a filter (e.g., an air filter, a chemical filter, an oil filter, an auto filter, etc.), packaging, an enclosure, inert material, fabric, a facemask, metal material, a solid, a liquid, and/or other suitable 3D objects. In some examples, the 3D object 42 may be considered a substrate for the coated carrier 40.

The coated carriers 40 may be positioned on or at the 3D object 42 randomly and/or to form one or more patterns or designs at any desired portion(s) of the 3D object 42. Example configurations of the coated carriers 40 applied to the 3D object 42 include, but are not limited to, bars, circles, lines, crosses, wording, lettering, numbers, punctuation, pictures, artistic designs, diagonals, grid patterns of rows and columns, concentric rings, color matching of a color of reactant material 22 with a color of the 3D object 42 prior to interactions with analyte, patterns that result in identifiable shapes when the reactant material reacts to a particular analyte, other suitable configurations, and/or combinations thereof. In one example, two or more coated carriers 40 with two different reactant materials 22 may be applied to the 3D object 42 in alternating fashion, but other suitable configurations are contemplated.

The coated carrier 40 may be applied to the 3D object 42 in any suitable manner. For example, the coated carrier 40 may be sprinkled onto the 3D object 42, applied to the 3D object 42 with adhesive (e.g., via glue, ultraviolet (UV) curing adhesives, and/or otherwise adhered to the multi-dimensional object), printed (e.g., 3D printed) onto the 3D object 42, and/or applied to the 3D object 42 in other suitable manners. The coated carrier 40 may be fixed relative to the 3D object 42 and/or loose or movable relative to the 3D object 42 once the coated area is applied to the 3D object 42.

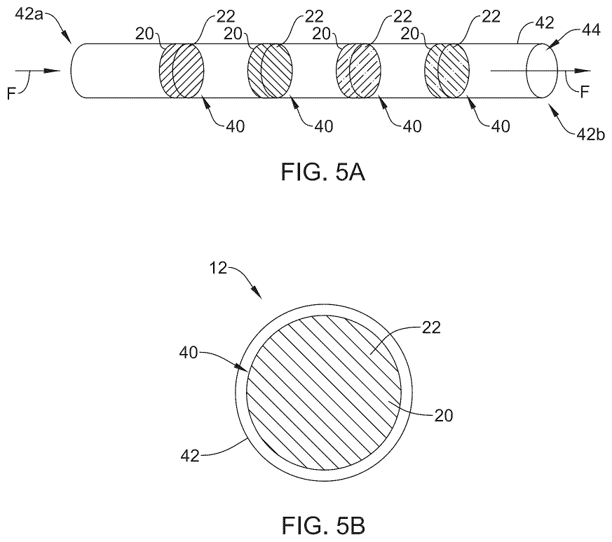

FIGS. 5A and 5B schematically depict a side view and an end view, respectively, of a reactant array 12, where the coated carrier 40 with the reactant material 22 on a sintered material 20 having disc shaped structures 30 are inserted into the 3D object 42 having a tube configuration (e.g., a flow tube) designed to receive a fluid for analysis at a first end 42a and output the fluid for analysis at a second end 42b (e.g., where the fluid may travel in the direction of the arrows F and/or in another suitable direction). To facilitate passing fluid through the 3D object 42 having one or more coated carriers 40 therein, the sintered material 20 of the coated carrier 40 may include pores with a suitable size such that after application of the reactant material 22 to the sintered material 20, there is sufficient space for the fluid to be analyzed to pass through the pores. Although the entirety of the sintered material 20 of each coated carrier 40 is covered with a single reactant material 22, it is contemplated that only a portion of the sintered material 20 may be covered with the reactant material 22 or multiple reactant materials 22 may be applied to a single structure 30 of sintered material 20.

When the 3D object 42 takes on a flow tube design, the flow tube may be positioned within a cartridge and the reactant array 12 thereof may be configured to be viewable by the reader device 16 through a transparent portion of the cartridge. Alternatively or additionally, the flow tube with one or more coated carriers 40 may be utilized independent of a cartridge.

The reactant array 12 may include any suitable number of coated carriers 40. In the example depicted in FIGS. 5A and 5B, the reactant array 12 may include four coated carriers 40, each with a different reactant material 22 applied thereto. In some examples, a number of coated carriers 40 and/or a number of reactant materials utilized may depend on what types of analytes are to be detected in the fluid passing through or along the reactant array 12.

Although the coated carriers 40 in FIGS. 5A and 5B are configured as discs and span an entire diameter of the tubular 3D object 42 (see FIG. 5B), the coated carriers 40 (e.g., the sintered material 20) may take on one or more other suitable shapes configured for exposure to fluid flowing through a lumen 44 of the tubular 3D object 42. Other example configurations include, but are not limited to, ring-shaped coated carriers 40, strip-like coated carriers 40 at a same or different axial locations of the tubular 3D object 42, dots, lines, and/or other suitable shapes and/or configurations.

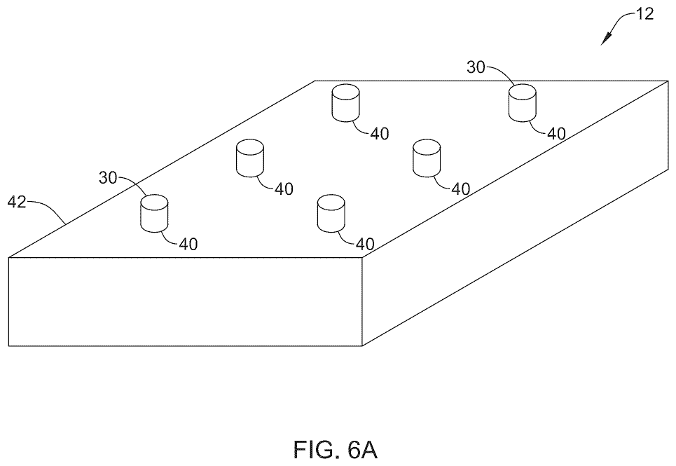

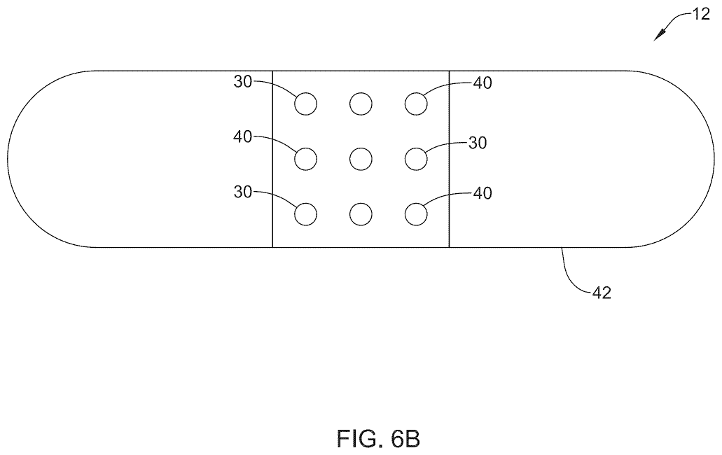

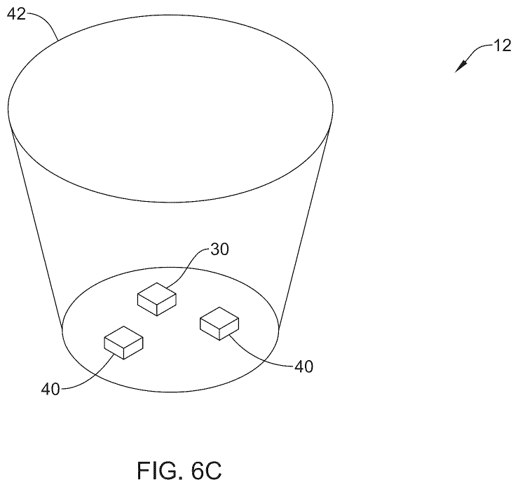

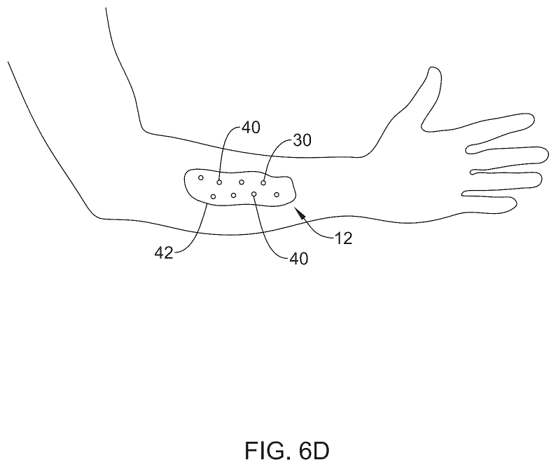

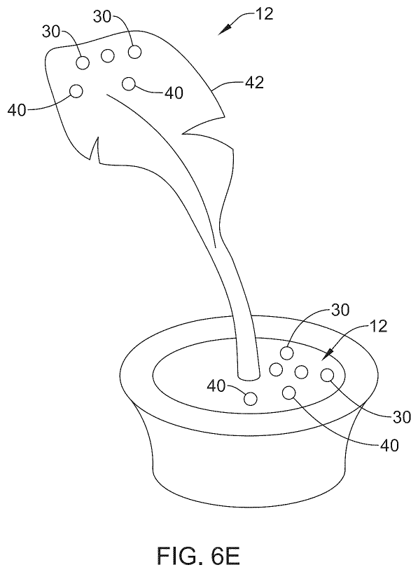

FIGS. 6A-6E schematically depict illustrative configurations of the reactant array 12 with coated carriers 40 with structures 30 applied to 3D objects 42 (e.g., substrates) having different shapes and/or configurations. For clarity purposes, not all coated carriers 40 with structures 30 are labeled. In some configurations, the 3D object 42 may be or include a filter (e.g., when configured as a substrate, a bandage, a filter product, etc.) or may be at least partially transparent to facilitate light traveling to and/or from the reactant material 22 of the coated carriers 40. Other suitable configurations for the reactant array 12 are contemplated.

FIG. 6A schematically depicts an illustrative configuration of the reactant array 12 with the 3D object 42 in a 3D rectangular shape, which may be a substrate of test strip, microscope slide, and/or other suitable rectangular shape. The coated carriers 40 used for detecting analyte in fluid passing over and/or through the 3D rectangular shape may have any suitable structure 30, including, but not limited to, structures 30 having circular cross-sections. The configuration of the reactant array 12 depicted in FIG. 6A may be utilized in a flow cell cartridge for exposure to a fluid and/or in other suitable configurations.

FIG. 6B schematically depicts an illustrative configuration of the reactant array 12 with the 3D object 42 configured as a bandage and coated carriers 40 applied to a portion 46 of the bandage configured for placement at a wound. The coated carriers 40 on the bandage may utilize any suitable structure 30, including granular or powdered particle structures 30. Although not required, a back side of the portion 46 of the bandage configured for placement at a wound may be transparent to facilitate the reader device 16 collecting light from the coated carriers 40 (e.g., the coated carriers 40 having granular or particle structures 30 and/or other suitable structures 30) as the coated carriers 40 are exposed to fluids from the wound to provide a fluid analysis in real time while a subject is wearing the bandage. Illustrative wounds may include, but are not limited to, cuts, rashes, infections, burns, and/or other wounds that may emit fluids. Further, the bandage may be placed over non-wound target areas that may or may not be associated with an animal subject (e.g., mammalian subjects, human subjects, and/or other animal subjects).

FIG. 6C schematically depicts an illustrative configuration of the reactant array 12 with the 3D object 42 in the form of a cup (e.g., a sample testing cup and/or other suitable cup). Although not depicted, the reactant array 12 may be located on or at a lid of a cup or a lid configured to couple with a cup, such that the reactant array 12 may not be submerged in a sample (e.g., a liquid sample), but is still able react to fluids from the sample.

The coated carriers 40 used for detecting analyte in fluid within the cup may have any suitable structure 30, including powder or granular particle structures 30 and may be applied to the cup in any suitable configuration to form the reactant array 12. For example, the coated carriers 40 applied to the cup may fill the cup entirely or at least partially, may be applied to the cup at random, may be applied to the cup with a particular design, may be applied to the cup so as to be fixed or loose or movable relative to the cup, and/or configured in one or more other suitable manners. The configuration of the reactant array 12 depicted in FIG. 6C may be configured to receive urine, consumable liquids, solids, and/or other samples for fluid analysis (e.g., urinary tract infection testing, drug testing, metabolic testing, poison testing, etc.)

FIG. 6D schematically depicts an illustrative configuration of the reactant array 12 with the 3D object 42 being a wound on an arm 48 of a subject. The coated carriers 40 used for detecting analyte in fluid from the wound may have any suitable structure 30, including powder or granular particle structures 30. The configuration of the reactant array 12 depicted in FIG. 6D may be viewable by the reader device 16 for collecting light from the coated carriers 40 as the coated carriers 40 are exposed to fluids from the wound to provide a fluid analysis in real time.

FIG. 6E schematically depicts two illustrative configurations of the reactant array 12 with the 3D object 42 being a leaf of a plant and the 3D object being soil in which the plant is planted. Although the plant in FIG. 6E may be living, the 3D object 42 may be a dead plant. The coated carriers 40 used for detecting analyte in fluid from the plant and/or from the soil may have any suitable structure 30, including powder or granular particle structures 30. The configuration of the reactant array 12 depicted in FIG. 6E may be viewable by the reader device 16 for collecting light from the coated carriers 40 as the coated carriers 40 are exposed to fluids from the leaf and/or the soil to provide a fluid analysis in real time.

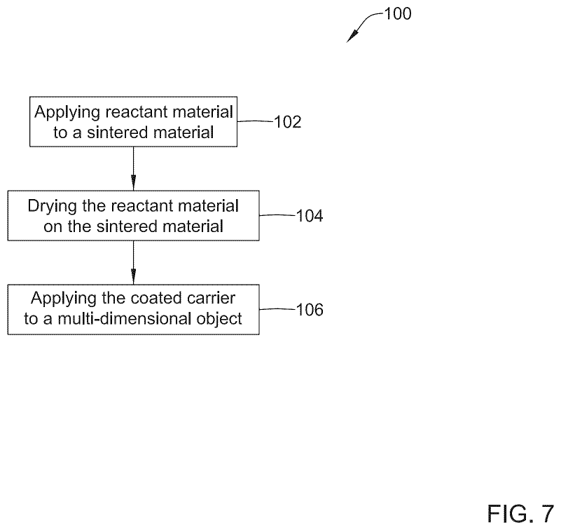

FIG. 7 schematically depicts an illustrative technique or method 100 of forming a reactant array. The method 100 may be applied to forming a reactant array having any suitable shape or configuration for any suitable application.

The method 100 may include applying 102 one or more reactant materials (e.g., chemical formulations) to one or more sintered materials. The reactant materials may be applied to the sintered material in any suitable manner. For example, the reactant material may be applied to the sintered material using one or more printing, deposition and/or coating techniques including, but not limited to, printing techniques, painting techniques, deposition techniques, electrostatic deposition techniques, physical vapor deposition techniques, vapor deposition techniques, soaking techniques, dipping techniques, brushing techniques, trickle techniques, spraying techniques, impregnating techniques, and/or other suitable techniques. In some examples, the reactant material may be applied to the sintered material as a powdered material and/or as an aqueous chemical solution (e.g., to facilitate covering at least part of or entire surface areas of the sintered material). As discussed above, the reactant material may be applied to the sintered material randomly and/or to form one or more patterns.

In one example, the reactant material may be applied to the sintered material by printing the reactant materials on the sintered material. When printed, any suitable printing techniques may be utilized including, but not limited to, pin transfer, inkjet, silkscreen, and/or other suitable application techniques.

In one example, the reactant material may be applied to the sintered material using an electrostatic deposition technique. Using an electrostatic deposition technique, a first charge may be applied to the reactant material (e.g., an aqueous chemical formulation of the reactant material) and a second, opposite charge may be applied to the sintered material, such that the reactant material is attracted to the sintered material in such a manner that the reactant material coats an entirety of or a desired portion of the sintered material.

In one example, the reactant material may be applied to the sintered material using an impregnating technique. In some examples, the impregnating technique may be a vacuum pressure technique, which may include cleaning and drying (e.g., with a vacuum or other suitable dryer) the sintered material, applying the reactant material to the sintered material, and applying one or more cycles of desired pressure(s) and temperature(s) to the sintered material for a suitable time. Other suitable impregnating techniques for applying reactant material to the sintered material are contemplated.

Once the reactant material has been applied to the sintered material, the reactant material on the sintered material may be dried 104. Once the reactant material is dried, a precipitate of the reactant material may remain on the sintered material that may change one or more properties when exposed to one or more analytes in a fluid. The dried reactant material on the sintered material may form a coated carrier.

Once the coated carrier is formed, the coated carrier may be applied 106 to a 3D object to form the reactant array, where the 3D object has any suitable configuration as discussed herein or otherwise. Alternatively, the 3D object and the step of applying the coated carrier to the 3D object may be omitted, such that the reactant array may be formed from the coated carrier.

As discussed herein, the coated carrier may be applied to the 3D object in any suitable manner including, but not limited to, by sprinkling the coated carrier onto the 3D object, applying the coated carrier to the 3D object with adhesive (e.g., via glue, ultraviolet (UV) curing adhesives, and/or otherwise adhered to the multi-dimensional object), printing (e.g., 3D printing) the coated carrier onto the 3D object, and/or applying the coated carrier to the 3D object in other suitable manners, where the coated carrier may be fixed relative to the 3D object and/or loose or movable relative to the 3D object once the coated area is applied to the 3D object. Further, the coated carrier may be applied to the 3D object randomly and/or to form one or more patterns.

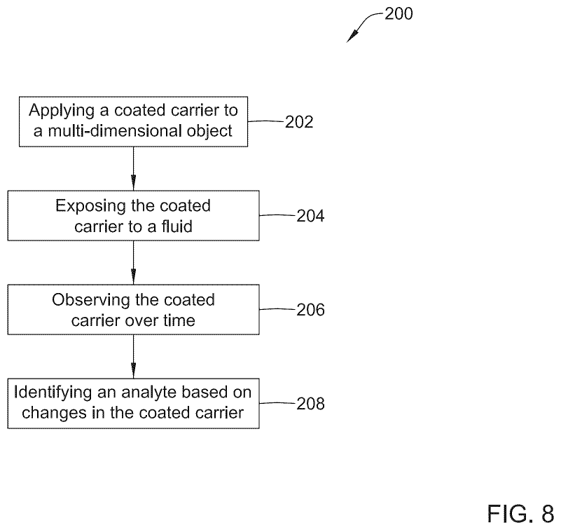

FIG. 8 schematically depicts an illustrative method 200 of analyzing or testing a fluid of interest for one or more analytes. The illustrative method 200 may be utilized for identifying bacteria in wounds, bacteria on plants or food, bacteria in sweat, bacteria or metabolites in urine, and/or for identifying other compounds or conditions, as discussed herein or otherwise.

The method 200 may include applying 202 one or more coated carriers to one or more multi-dimensional objects. As discussed herein, the multi-dimensional object(s) may be any suitable multi-dimensional object discussed herein or otherwise including, but not limited to, a tube, a membrane, a diaphragm, an elongated support, a cup, a bandage, a band (e.g., a headband, sweat band, etc.), skin of a subject (e.g., a human subject, mammalian subject and/or other animal subject), a wound of a subject, a living plant, a dead plant, a flower, soil, polymer material, a filter (e.g., an air filter, a chemical filter, an oil filter, an auto filter, etc.), packaging, an enclosure, inert material, fabric, a facemask, metal material, a solid, liquid, and/or other suitable 3D objects.

The coated carrier(s) may be applied to a multi-dimensional object in any suitable manner as discussed herein or otherwise. For example, and as discussed herein, the coated carrier(s) may be sprinkled onto the multi-dimensional object, applied to the multi-dimensional object with adhesive (e.g., via glue, ultraviolet (UV) curing adhesives, and/or otherwise adhered to the multi-dimensional object), printed (e.g., 3D printed) onto the multi-dimensional object, and/or applied to the multi-dimensional object in other suitable manners. The coated carrier(s) may be fixed relative to the multi-dimensional object and/or loose or movable relative to the 3D object once the coated area is applied to the 3D object. As discussed above, the coated carrier(s) may be applied to the multi-dimensional object randomly and/or to form one or more patterns.

Further, the method 200 may include exposing 204 the coated carrier(s) to a fluid of interest. In one example, when the 3D object is a flow tube and the coated carrier(s) are located in a lumen of the flow tube through which fluid passes, exposing the coated carrier(s) to a fluid may include actively exposing the coated carrier(s) to the fluid by applying the fluid through the lumen of the tube and through the coated carrier(s). In one example, when the 3D object is a plant, exposing the coated carrier(s) to a fluid may include passive exposing the coated carrier(s) to the fluid over time as the plant releases fluid. Other suitable techniques for actively and/or passively applying fluids to the coated carrier(s) are contemplated.

The method 200 may include observing 206 the coated carrier(s) over time. The observing of the coated carrier(s) may occur before or after initial exposure of the coated carrier(s) to the fluid and/or while the coated carrier(s) are exposed to the fluid. In some configurations, observing the coated carrier(s) over time may include, but is not limited to, collecting light from the coated carrier. The light from the coated carrier(s) may be collected with a reader device using a light or image sensor and/or collected in one or more other suitable manners and may be indicative of one or more changes in properties of the coated carrier(s) in response to exposure to the fluid. Alternatively or additionally, a user, without a reader device (e.g., via the naked eye or the naked eye with one or more lenses) may observe the coated carrier(s) for changes in one or more properties thereof over time before, during, or after exposure of the coated carrier(s) to the fluid.

In some configurations, the reader device may be configured to analyze levels of wavelengths of light collected from the reactant array. In some examples, the levels of the wavelengths of light collected from the reactant array may be analyzed by measuring the light collected in any suitable manner including, but not limited to, by counting photons at one or more wavelengths of light collected, measuring an amount of light collected at one or more wavelengths of light collected, a change in photon count over time for one or more wavelengths of light collected, a change in pixel value (e.g., a change in pixel grayscale value) of an image sensor over time, and/or levels of wavelengths of light collected may be measured in one or more other suitable manners.

Further, the method 200 may include identifying 208 an analyte of the fluid to which the coated carrier(s) is exposed based on a change in a property of one more properties of one or more reactant materials of the coated carrier(s) in response to the coated carrier(s) being exposed to the fluid. In some examples, when the measurements of levels of light are collected while observing the coated carrier(s), the measurements of the levels of the wavelengths of light collected from the coated carrier(s) may be utilized to identify a component of the fluid to which the reactant array may be exposed. In some examples, when the levels of the wavelengths of light collected over time match or closely resemble a set of expected wavelength levels of the coated carriers (e.g., reactant material thereof) exposed to a known analyte (e.g., known fluid or component of fluid), the known analyte may be identified as an analyte in the fluid tested in the fluid analysis test. Example techniques for measuring levels of wavelength of light collected from reactant arrays and for comparing measurements to known measurements associated with fluids are discussed in PCT Application No. PCT/US2023/083024 (Attorney docket no. 1519.1004111), titled DEVICES, METHODS, AND SYSTEM FOR MEASURING AND RECORDING SPECTRUM OF A REACTANT ARRAY, having the same filing date as this application, which has been incorporated by reference herein. Other suitable techniques for identifying an analyte of the fluid to which the coated carrier(s) is exposed are contemplated.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless otherwise expressly stated, it is in no way intended that any method or technique set forth herein is to be construed as requiring that its steps be performed in a specific order. This holds for any possible non-express basis for interpretation, including matters of logic with respect to arrangement of steps or operational flow, plain meaning derived from grammatical organization or punctuation, and the number or type of embodiments described in the specification

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A reactant array comprising:
a sintered material; and
a reactant material applied to the sintered material, and
wherein the reactant material is configured to change one or more properties in response to exposure to one or more analytes.

2. The reactant array of claim 1, wherein the reactant material is applied to an entirety of a surface area of the sintered material.

3. The reactant array of claim 1, wherein the reactant material is applied to a desired portion of a surface area of the sintered material.

4. The reactant array of claim 1, further comprising:
a substrate, and
wherein the sintered material and the reactant material applied to the sintered material form a coated carrier and the coated carrier is secured to the substrate.

5. The reactant array of claim 4, wherein the substrate is a filter.

6. The reactant array of claim 1, further comprising:
a three-dimensional object, and
wherein the sintered material and the reactant material applied to the sintered material form a coated carrier and the coated carrier is applied to the three-dimensional object.

7. The reactant array of claim 6, wherein the three-dimensional object comprises a bandage.

8. The reactant array of claim 6, wherein the three-dimensional object comprises a living plant.

9. The reactant array of claim 6, wherein the three-dimensional object comprises a sample testing cup.

10. The reactant array of claim 6, wherein the three-dimensional object comprises a filter.

11. A method of forming a reactant array, comprising:
applying a reactant material to a sintered material;
drying the reactant material on the sintered material to form a coated carrier; and
applying the coated carrier to a multi-dimensional object.

12. The method of claim 11, wherein the applying the reactant material to the sintered material includes applying the reactant material to the sintered material using an electrostatic deposition technique.

13. The method of claim 11, wherein the applying the reactant material to the sintered material includes impregnating the sintered material with an aqueous chemical solution.

14. The method of claim 11, wherein the multi-dimensional object comprises a bandage.

15. The method of claim 11, wherein the multi-dimensional object comprises a living plant.

16. The method of claim 11, wherein the multi-dimensional object comprises a sample testing cup.

17. The method of claim 11, wherein the multi-dimensional object comprises a sample testing cup.

18. A method of testing fluid, comprising:
applying a coated carrier to a multi-dimensional object;
exposing the coated carrier to a fluid;
observing the coated carrier over time after initial exposure of the coated carrier to the fluid; and
identifying an analyte of the fluid based on a change in a property of one or more properties of the coated carrier in response to exposure to the fluid.

19. The method of claim 18, wherein the coated carrier comprises a sintered material to which a reactant material is applied.

20. The method of claim 18, wherein the multi-dimensional object comprises skin on a subject.

21. The method of claim 18, wherein the multi-dimensional object comprises a bandage configured to be applied to a wound of a subject.

22. The method of claim 18, wherein the multi-dimensional object comprises a sample testing cup.

23. The method of claim 18, wherein applying the coated carrier to the multi-dimensional object comprises sprinkling the coated carrier on the multi-dimensional object.
